(19) United States
(12) Patent Application Publication
(10) Pub. No.: US 2023/0392063 A1
(43) Pub. Date: Dec. 7, 2023

(54) BIOCHEMICAL REACTION TEMPERATURE REGULATOR WITH LIGUID-GAS PHASE CHANGE MATERIAL

(71) Applicant: DOMUS DIAGNOSTICS, INC., Park City, UT (US)

(72) Inventor: Joshua R. BUSER, Park City, UT (US)

(21) Appl. No.: 18/205,992

(22) Filed: Jun. 5, 2023

Related U.S. Application Data
(60) Provisional application No. 63/349,233, filed on Jun. 6, 2022.

(51) Int. Cl. C09K 5/04; C12Q 1/6844

(52) U.S. Cl. CPC C09K 5/048; C12Q 1/6844

(57) ABSTRACT

Disclosed is a temperature regulation device and system configured to regulate temperature in chemical reactions, in particular biochemical reactions. The temperature regulation device includes a film arranged to form a sealed pouch. The sealed pouch encloses a phase change material (e.g., a solvent formulated with a desired boiling point), which may be optionally held by a solid carrier. The temperature regulation device described herein can be combined with a heat source and a reaction chamber, such as a reaction chamber associated with a reaction chip or card, to form a temperature regulation system.

### CROSS-REFERENCE TO RELATED APPLICATIONS

This application claims priority to and the benefit of U.S. Provisional Patent Application No. 63/349,233, filed on Jun. 6, 2022, the entirety of which is incorporated herein by reference.

### BACKGROUND

Certain chemical reactions must be performed within specified temperature range to be accurate and/or effective. In particular, biochemical reactions and reactions relating to biomaterials may be sensitive to temperature changes. In certain instances, the bio-samples themselves may denature or become damaged when the temperature fluctuates. Further, depending on the process being performed, the technique itself may be temperature dependent and may be less efficient or ineffective if exposed to excessive temperature fluctuations.

As one example, loop-mediated isothermal amplification (LAMP) assays have been developed to provide diagnostic tests which are a low-cost alternative to polymerase chain reaction (PCR) techniques. One specific variation of the LAMP technique is reverse transcription loop-mediated isothermal amplification (RT-LAMP).

Both LAMP and RT-LAMP reactions must typically be performed at a temperature of about 60 to about 70 degrees Celsius. When performed in the lab, temperatures can be monitored and relatively easily controlled. Outside the lab, however, such as with home or field testing, monitoring the temperature may be challenging or impossible. In these settings, temperature variations are common and can be detrimental to accurate diagnostic testing.

In sum, regulating temperature in chemical reactions is often necessary but sometimes not possible under certain conditions. As more techniques are developed that are intended for use outside of a laboratory setting, temperature regulation has turned into a larger challenge for accurate testing and assay control. Accordingly, there is a long felt and ongoing need for efficient, low-cost, and high-performance means for regulating temperature in chemical reactions, in particular biochemical reactions such as LAMP and RT-LAMP.

### SUMMARY

The present disclosure relates to a temperature regulation device, a temperature regulation system, and related methods. Embodiments described herein solve one or more technical problems related to regulating temperature in chemical reactions, and in particular, biochemical reactions. For example, when utilizing LAMP or RT-LAMP to amplify nucleic acid sequences, the temperature is preferably maintained at about 60 to about 70 degrees Celsius. The temperature regulation device described herein can be used in conjunction with one or more biochemical reaction chambers, such as one or more LAMP or RT-LAMP reaction chambers, and a heat source to create a temperature regulation system for effectively regulating temperature while the reaction is carried out.

An example temperature regulation device includes a film arranged to form a sealed pouch. The sealed pouch encloses a phase change material (e.g., a solvent formulated with a desired boiling point), which may be optionally held by a solid carrier. The solid carrier is configured to fit within the pouch and can beneficially help distribute the phase change material to minimize the formation of hot or cold spots during use of the temperature regulation device. The temperature regulation device described herein can be combined with a heat source and a reaction chamber to form a temperature regulation system.

In some embodiments, the reaction chamber is a LAMP or RT-LAMP reaction chamber, such as a LAMP or RT-LAMP “card” or “chip” useful for remote (i.e., outside of the laboratory) assaying. In some embodiments, the heat source is an electronic heater or an exothermic reaction device. In use, the temperature regulation device can be disposed between the heat source and the reaction chamber. Upon activation of the heat source, the heat source will heat the temperature regulation device, including the phase change material within the temperature regulation device (which is optionally held by the solid carrier). In the simplest explanation, when the temperature reaches the boiling point of the phase change material, a liquid to gas phase transition occurs. This can cause the temperature regulation device to begin to expand. While the phase change material undergoes phase change, the temperature within the sealed pouch will be maintained at the boiling point and can therefore beneficially buffer against temperature fluctuations inherent in the heat source. The overall composition of the contents inside of the temperature regulator pouch determine the thermal conductivity of the pathway between the heat source and the reaction chamber(s).

While contacted with the reaction chamber, the temperature regulation device functions to maintain the reaction chamber at a temperature substantially near the boiling temperature of the phase change material included in the temperature regulation device. Even if the heat source provides inconsistent or fluctuating heat, which is particularly common when using a heat source suitable for remote applications, the temperature regulation device functions as a buffer between the heat source and the reaction chamber to provide a substantially consistent temperature to the reaction chamber so long as the heat source remains above the lowest permissible temperature for the assay.

The liquid-gas transition of the contents of the temperature regulator determines how it responds to applied temperature from the heat source. The solvent may evaporate at temperatures lower than the boiling point at certain atmospheric pressures, and multiple solvents may be used to tune the gas-liquid phase change response to temperature. Different embodiments may use different phase change material formulations as determined by the desired regulated temperature, which can be determined based on the specific chemical reaction being performed.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIGS. 1A and 1B illustrate an example of a temperature regulation device, with FIG. 1B schematically showing a cross-sectional view to illustrate interior components of the device.

FIG. 1C shows a phase change diagram for a binary solvent phase change system composed of methanol and ethanol;

FIGS. 2A and 2B illustrate an example of a solid carrier being inserted into a pouch to form the temperature regulation device;

FIG. 3 illustrates a side view of the temperature regulation device in a heated/expanded state;

FIGS. 4A and 4B illustrate an example temperature regulation system that includes the temperature regulation device of FIGS. 1-3, a heat source, and a reaction chamber, with FIG. 4A showing an exploded view of the system and FIG. 4B showing the assembled system;

FIG. 5 illustrates experimental results showing effective use of the temperature regulation device of FIGS. 1-3 to regulate heat generated by an electric heater;

FIG. 6 illustrates experimental results showing effective use of the temperature regulation device of FIGS. 1-3 to regulate heat generated by an exothermic reaction device;

FIG. 7 is a temperature response plot comparing multiple temperature regulation devices with different formulations of phase change materials;

FIGS. 8A and 8B are temperature response curves for temperature regulation devices including denatured alcohol and methanol, respectively; and

FIG. 9 plots the temperature of an integrated biochemical reaction device with a temperature regulation system over time, showing that the tested temperature regulation system effectively regulated the temperature within a target temperature range for an extended time period.

### DETAILED DESCRIPTION

### Introduction

The embodiments described herein can solve one or more of the problems associated with temperature regulation of biochemical reactions discussed above. The embodiments described herein are particularly useful for low cost and/or remote applications where power and/or standard laboratory equipment is not readily available or is not economically feasible.

As explained in greater detail below, the temperature regulation device, and temperature regulation systems incorporating such a device, may be used to regulate temperature in a variety of applications such as in temperature sensitive biochemical reactions. One example of a temperature sensitive biochemical reaction is a LAMP or RT-LAMP reaction. Such reactions typically have a target temperature of between about 60 and about 70 degrees Celsius.

The disclosed temperature regulation devices may also be utilized in other isothermal amplification reactions, such as rolling circle amplification (RCA). RCA typically has a target temperature of about 30 to about 45 degrees Celsius, though in some instances it may be performed at even lower temperatures.

When in a laboratory setting, temperature is readily regulated. However, when performing LAMP or RT-LAMP based assays, or other biochemical reactions, in a home or field setting, regulating the temperature may be challenging. As used herein, a “remote” setting will refer to any environment in which finely controlled, laboratory-grade temperature regulation is not available and/or is not convenient. Examples include a home setting, a field setting, an outdoor setting, a setting where power supply is not readily available, a temporary “pop-up” clinic setting, a mobile medical unit, or even a healthcare facility where cost or operator training limitations reduce the feasibility of providing biochemical reactions with laboratory-grade temperature regulation.

As discussed above, certain biochemical reactions require a specified temperature range to be effectively carried out. With LAMP or RT-LAMP, for example, the temperature is preferably maintained at about 60 to about 70 degrees Celsius. The temperature regulation device described herein can be used in conjunction with one or more biochemical reaction chambers, such as one or more LAMP or RT-LAMP reaction chambers, and a heat source to create a temperature regulation system for effectively regulating temperature while the reaction is carried out.

### Example Temperature Regulation Device

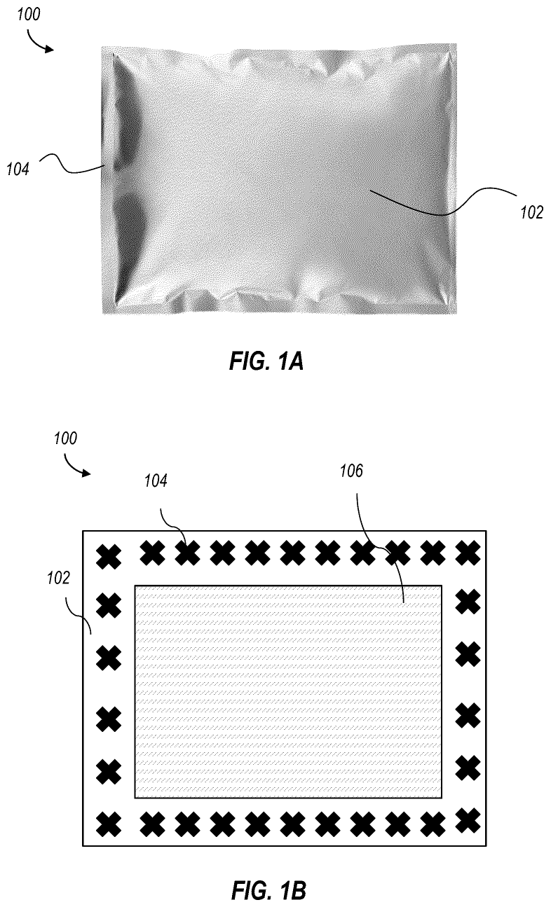

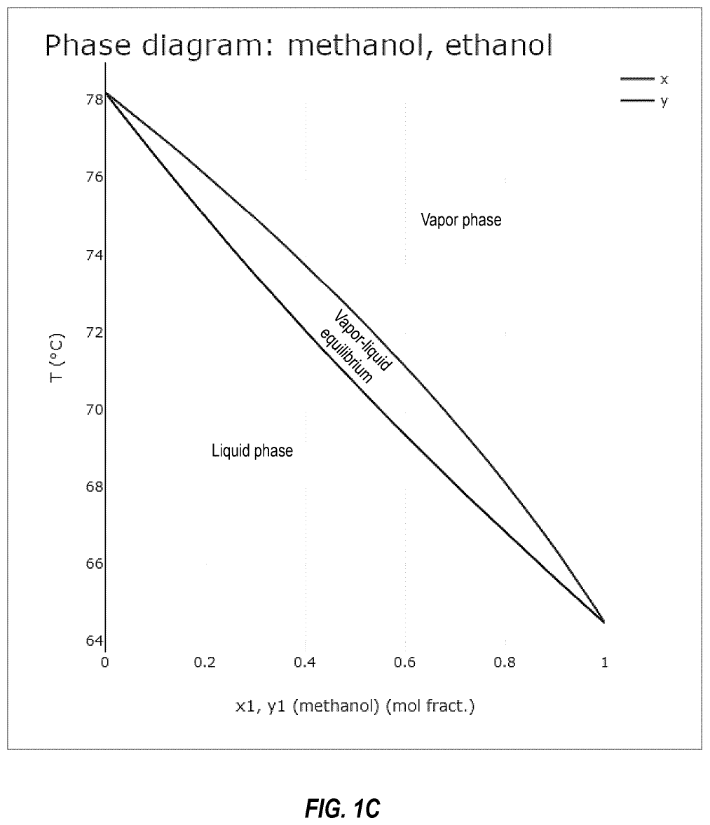

FIGS. 1A and 1B illustrate an example of a temperature regulation device 100, with FIG. 1B schematically showing a cross-sectional view to illustrate interior components of the device 100. The illustrated temperature regulation device 100 includes a film 102 and a solid carrier 106.

The film 102 may include a polyester-based film such as a polyethylene terephthalate (e.g., biaxially oriented polyethylene terephthalate (BoPET), known by the trade name MYLAR), polypropylene, nylon, polyethylene, and/or polypropylene, and may additionally or alternatively include other materials known in the art as suitable for film applications. For example, the film 102 may be additionally or alternatively made of other polyester materials and/or other polymer materials appropriate as films for a temperature regulation device. The film 102 may be metalized. A metalized film includes a thin coating of metal, typically aluminum, though other metals such as nickel and/or chromium may be utilized.

Although most embodiments are described herein as a “film,” it will be understood that other embodiments may additionally or alternatively utilize other structures capable of providing sufficient structural integrity to house a solvent and sufficient heat-transfer capacity to heat an adjacent reaction chamber.

The solid carrier 106 may be formed of a suitable fabric, such as a poly broadcloth, a polyester/cotton blend fabric, and/or other fabric material capable of holding a solvent phase change material. In some embodiments, the solid carrier 106 may additionally or alternatively include (e.g., non-fabric) solid materials such as, for example, absorbent beads, granular materials, sponge materials, paper or other cellulose material, fibrous materials, fiber bundles, or combinations thereof. Although a solid carrier 106 is presently preferred, other embodiments may omit a solid carrier 106 and simply include a phase change material directly added to the inner cavity of the film 102.

The solid carrier 106, when included, can be sized to substantially match a planar surface area of the inner cavity of the device 100. For example, the solid carrier 106 can be sized to equal at least 50%, at least 60%, at least 70%, at least 80%, at least 85%, at least 90%, or at least 95% (or a range using any two of the foregoing as endpoints) of the planar surface area of the inner cavity of the device 100.

The “planar surface area” of the inner cavity is the projected area of the inner cavity from a plan view perspective (i.e., from the perspective shown in FIG. 1B). That is, the planar surface area is equal to the length of the inner cavity multiplied by the width of the inner cavity (i.e., the two longest dimensions of the inner cavity). The planar surface area is also the effective surface area from which the reaction chamber receives heat during use.

As shown in FIG. 1B, the film 102 can form a pouch where the edges are sealed using a heat seal 104. In other embodiments, the pouch may additionally or alternatively be sealed using adhesive, thread, staples, stitching, folding, and/or other methods appropriate for forming a pouch out of a film.

The illustrated embodiment shows four edges included in the heat seal 104. Other embodiments may have a different configuration. For example, a piece of film may be folded back on itself or formed as a circle, thus forming one or more “edges” of the pouch that will not require a heat seal 104.

The solid carrier 106 can be soaked or dampened with the phase change material. The phase change material may include, for example, an inorganic solvent and/or an organic solvent. Suitable solvents include methanol, n-hexane, cyclohexane, ethanol, ethyl acetate, isopropanol, tert-butanol, benzene, tetrahydrofuran, other solvents, or combinations thereof.

Generally, solvents are suitable where they exhibit relatively minimal toxicity and have boiling points at or slightly higher than the target temperature of the intended reaction. Solvent mixtures may also be utilized, particularly where the mixed solvents have similar boiling points. With solvent mixtures, one or more solvents of the mixture may exhibit a boiling point outside the target range of the intended reaction, but the overall temperature regulation effect of the mixture can still be effective for maintaining the reaction within the target temperature range.

The phase change material can be selected based on the desired regulated temperature of the system and the boiling point of the phase change material. In a presently preferred embodiment, the phase change material includes methanol, ethanol, isopropanol, or some combination thereof. Some formulations of denatured alcohol, for example, include ethanol in combination with smaller amounts of methanol (e.g., 1 wt. % to 15 wt. %) and/or isopropanol (e.g., 1 wt. % to 15 wt. %). Some embodiments may include a mixture of methanol and denatured alcohol, such as at a methanol to denatured alcohol ratio of 0.5:1 to 2:1, or 0.75:1 to 1.5:1, or 1:1, or a range of ratios with any two of the foregoing values as endpoints.

FIG. 1C is a phase transition diagram of an ethanol/methanol mixture as an example of a solvent mixture that may be utilized in the temperature regulation device 100. As shown, with ethanol, the liquid to gas transition happens at the boiling point of ethanol, which here is plotted just above 78 degrees Celsius. As more methanol is added (increasing methanol mol fraction is shown along X axis), the liquid to gas transition occurs at progressively lower temperatures, and a vapor-liquid equilibrium region occurs. With pure methanol, the transition happens at just above 64 degrees Celsius, the boiling temperature of methanol. This example illustrates that solvent mixtures can be formulated to tune the regulated temperature as needed for a particular assay.

In LAMP or RT-LAMP, the reaction is preferably carried out at a temperature of about 60 and about 70 degrees Celsius. Methanol has a boiling point of about 65 degrees Celsius. This results in methanol undergoing a liquid to gas phase change and therefore boiling at about 65 degrees Celsius. During the phase change, the temperature regulation device will thus regulate/maintain the temperature of the RT-LAMP reaction at about 65 degrees Celsius. Ethanol and Isopropanol have higher boiling points (about 78 degrees Celsius and about 82 degrees Celsius, respectively), but can still be utilized in at least some applications for effective temperature regulation within the target LAMP/RT-LAMP range of 60 to 70 degrees Celsius.

During use, the temperature of the reaction will tend to be slightly lower than the temperature within the temperature regulation device due to inherent heat transfer losses. Thus, in some implementations, the phase change material may be formulated with a boiling point that is slightly above the target temperature of the intended reaction. For example, the phase change material can be formulated with a boiling point that is 0.1, 0.5, 1, 2.5, 5, 7.5, 10, 12.5, or 15 degrees Celsius (or a range using any combination of the foregoing as endpoints) higher than a target reaction temperature. This intentional offset can account for such heat transfer effects and bring the reaction temperature closer to the intended target temperature.

In other embodiments, the temperature regulation device may need to regulate at other temperatures. For example, some biochemical reactions may have target temperatures of about 20 degrees Celsius, or 30 degrees Celsius, or 40 degrees Celsius, or 50 degrees Celsius, or 60 degrees Celsius, or 70 degrees Celsius, or 80 degrees Celsius, or 90 degrees Celsius, or even close to 100 degrees Celsius, or another value within a range defined by any two of the foregoing temperatures, and the phase change material can be selected accordingly.

The amount of phase change material added to the solid carrier 106 may vary. For example, the temperature regulation device 100 can include about 0.01 mL, or 0.05 mL, or 0.1 mL, or 0.15 mL, or 0.2 mL, or 0.25 mL, or 0.5 mL, or 0.75 mL, or 1.0 mL, or 1.25 mL, or 1.5 mL, or 1.75 mL, or 2.0 mL, or 2.25 mL, or 2.5 mL of phase change material, or any value within a range defined by any two of the foregoing volumes.

The amount of phase change material may also vary according to the overall size of the temperature regulation device. For example, the temperature regulation device may have a planar surface area (e.g., from the surface that contacts the reaction chamber) of about 10 to about 100 square centimeters, or about 20 to about 80 square centimeters, or about 30 to about 60 square centimeters. Certain embodiments may be, for example, within about 0.5 to about 10 square centimeters or about 5 to about 25 square centimeters. The temperature regulation device may be sized with a planar surface area within a range using any two of the foregoing values of this paragraph as endpoints. The foregoing phase change material volumes may apply to a similarly sized temperature regulation device and may be scaled accordingly for devices of other sizes.

Accordingly, the temperature regulation device 100 may include a phase change material volume to planar surface area ratio of about 0.05 μL/cm² to about 250 μL/cm², such as about 0.5 μL to about 225 μL, or about 1 μL to about 200 μL, or C about 2.5 μL to about 175 μL, or about 5 μL/cm² to about 150 μL/cm², or about 25 μL/cm² to about 125 μL/cm², or about 50 μL/cm² to about 100 μL/cm², or a ratio within a range using any two of the foregoing values as endpoints.

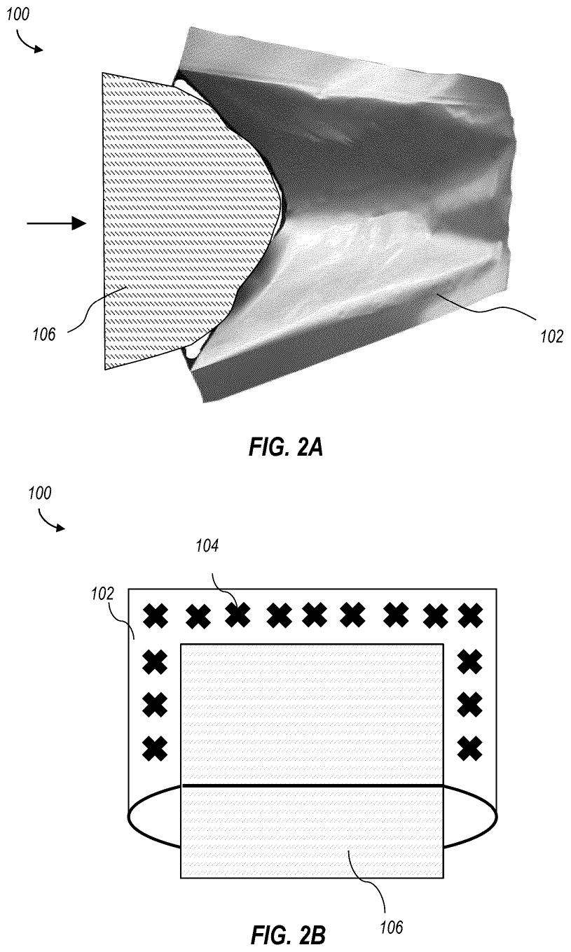

FIGS. 2A and 2B illustrate an example of the solid carrier 106 being inserted into the pouch formed by film 102. The phase change material can be added to the solid carrier 106 before or after the solid carrier 106 is placed within the pouch. The pouch can be fully sealed after placement of the solid carrier 106.

In some embodiments, the solid carrier 106 is formed into a rectangular shape. In other embodiments, the solid carrier 106 may be formed into an oval, circle, triangle, or other appropriate shape. The shape of the solid carrier 106 will most often correspond to the general shape of the pouch, but the solid carrier 106 and pouch need not necessarily have the same shape.

In some embodiments, the solid carrier 106 may have a length of about 10 mm, or 20 mm, or 30 mm, or 50 mm, or 70 mm, or 80 mm, or 90 mm, or 100 mm, or any value within a range defined by any two of the foregoing lengths. In these embodiments, the solid carrier 106 may have a width of about 30 mm, or 40 mm, or 50 mm, or 60 mm, or 80 mm, or 90 mm, or 100 mm, or 110 mm, or 120 mm, or any value within a range defined by any two of the foregoing widths.

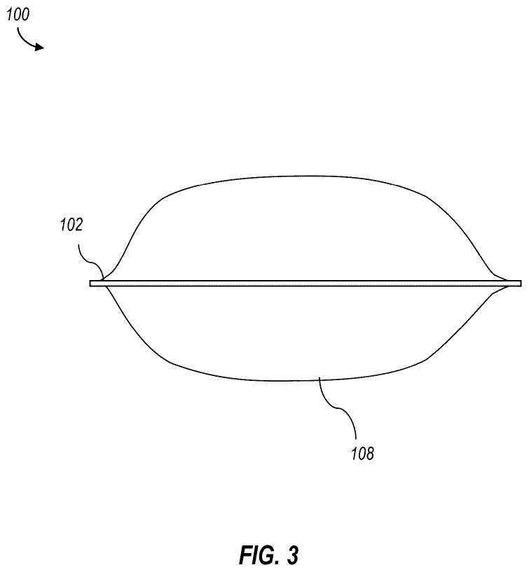

FIG. 3 illustrates a side view of the example temperature regulation device 100 formed of a film 102 with an expanded pouch 108. When the phase change material is heated at or above its boiling point, the phase change material undergoes a liquid-gas phase change. When the phase change occurs and the material changes to the gas phase, the gas takes up more space and can result in expansion of the pouch 108.

It will be understood that the amount of pouch expansion shown in FIG. 3 is illustrative, and other implementations may involve different levels of expansion when used. For example, at least some applications may involve expansion of about 0.1 to 5 mm, or about 2 to 10 mm, or about 5 to 25 mm, or an amount of expansion within a range with endpoints defined by any two of the foregoing values. The amount of expansion refers to the difference in the height of the pouch prior to heating and during or following heating.

### Example Temperature Regulation System

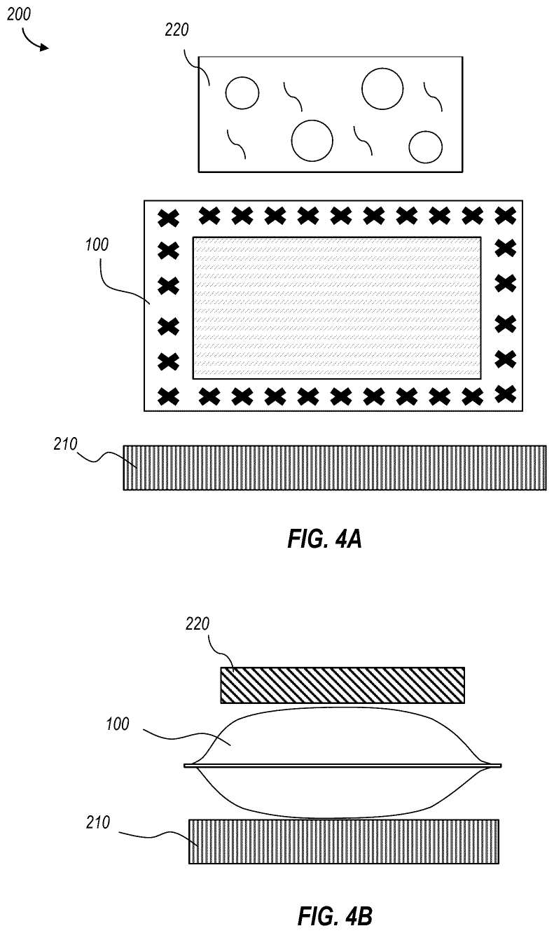

FIG. 4A illustrates an exploded view of an example temperature regulation system 200 including the temperature regulation device 100, a heat source 210, and a reaction chamber 220. In some embodiments, the heat source 210 includes an electric heater. In many embodiments, however, the heat source 210 includes a heat source that does not require electrical power to provide heat, such as a heat source that relies on an exothermic reaction to generate heat. Example exothermic reaction devices include oxygen reactive devices (e.g., devices based on reaction of ambient air with iron and/or zinc), water reactive devices (e.g., devices based on a reaction of water and sodium chloride with magnesium and iron) and crystallization/nucleation-based devices (e.g., supersaturated sodium acetate devices).

The reaction chamber 220 may be a biochemical reaction chamber such as an isothermal nucleic acid amplification chamber, including a LAMP amplification chamber, an RT-LAMP amplification chamber, or other reaction chamber that requires temperature regulation. The reaction chamber 220 may be included as part of a “chip” or “card” useful for remote settings.

FIG. 4B illustrates an example of the assembled temperature regulation system 200. Some embodiments may additionally include one or more insulation layers disposed around the components of the system 200. An insulation layer may include, for example, insulative foam (e.g., expanded polystyrene), mineral wool, fiberglass materials, cellulose materials, insulative fabrics, other suitable insulative materials, and combinations thereof.

The temperature regulation device 100 may be disposed between the heat source 210 and the reaction chamber 220. The heat source 210 and/or reaction chamber 220 may be attached to the temperature regulation device 100 using an adhesive (e.g., glue or tape) and/or mechanical fixation (e.g., clamping of the components together). In the illustrated example, the temperature regulation device 100 has expanded somewhat, implying that at least a portion of the phase change material has undergone a liquid to gas phase change.

### WORKING EXAMPLES

### Example 1

A temperature regulation device was created by using a metalized MYLAR film which was heat sealed on three sides to form a partially sealed pouch. A rectangular piece of fabric formed of poly broadcloth and polyester/cotton blend with dimensions of 50 mm by 80 mm was placed in the pouch and 1 mL of methanol was added thereto. Air was then evacuated from the pouch and the fourth side was heat sealed to create a fully sealed pouch.

In the experiments, two different heat sources were used: an electric heater and a “90C” oxygen-reactive pouch available from Exothermix (College Station, TX).

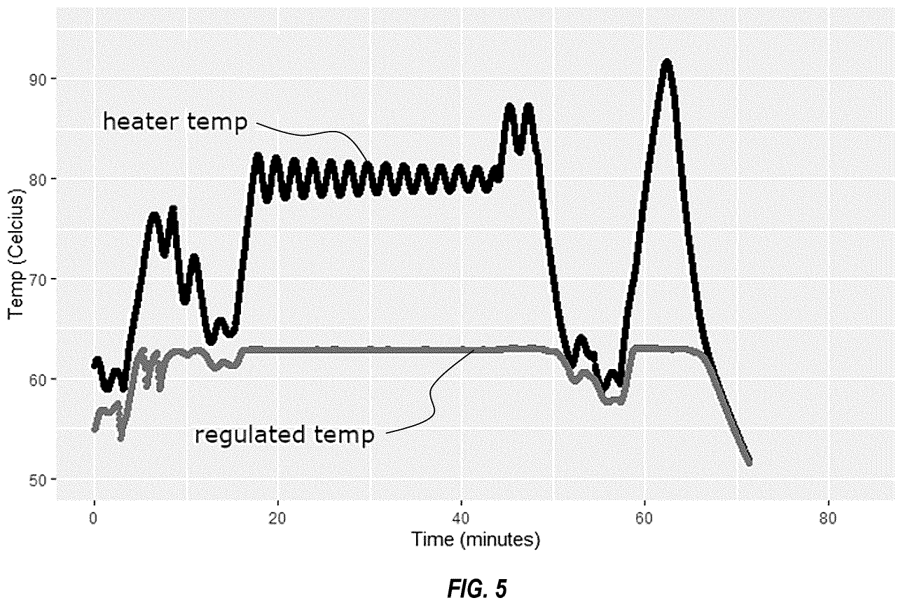

The tested temperature regulation system included a resistive electronic heater adhered to the temperature regulation device. The temperatures of (1) the electronic heater and (2) the temperature regulation device (on the side opposite the electronic heater) were measured for a duration of 70 minutes. The results of this test are plotted in FIG. 5.

Once the electronic heater exceeded temperatures above about 65 degrees Celsius, the temperature regulation device began to regulate/maintain temperature at about 63 degrees Celsius, even though the electronic heater reached 80 to 90 degrees Celsius. When the electronic heater was below about 65 degrees, the temperature regulation device had a temperature similar to the electronic heater.

### Example 2

In a second experiment, a temperature regulation device as in Example 1 was coupled to a “90C” oxygen-reactive pouch available from Exothermix (College Station, TX). The temperature of the temperature regulation device (on the side opposite the oxygen reactive pouch) was measured for a duration of 13 to 25 minutes over multiple runs.

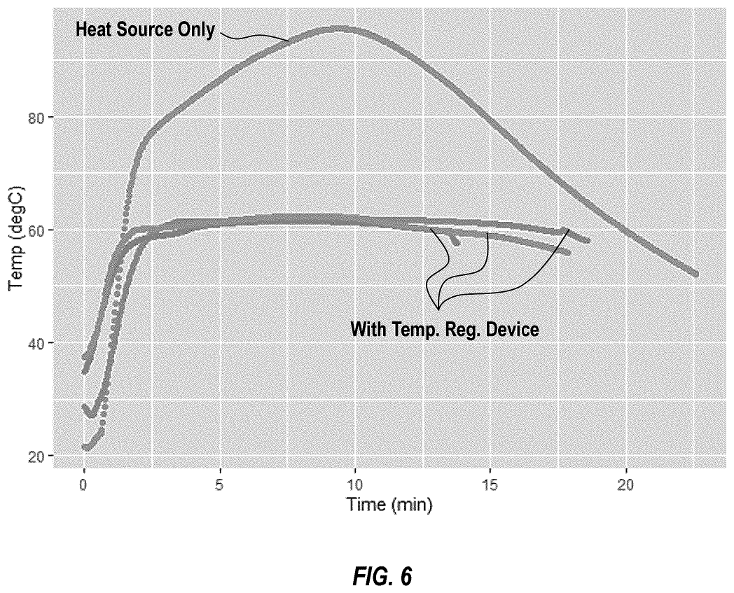

Following an initial test run, four additional runs were conducted, with one measuring the temperature of only the heat source without an attached temperature regulation device, while the other three runs measured the temperature of the temperature regulation device coupled to the heat source. The results are plotted in FIG. 6.

As shown, the “90C” oxygen-reactive pouch reached temperatures slightly above 85 degrees Celsius. When coupled with the temperature regulation device, the temperature was effectively regulated to just above about 60 degrees Celsius in all three of such runs.

### Example 3

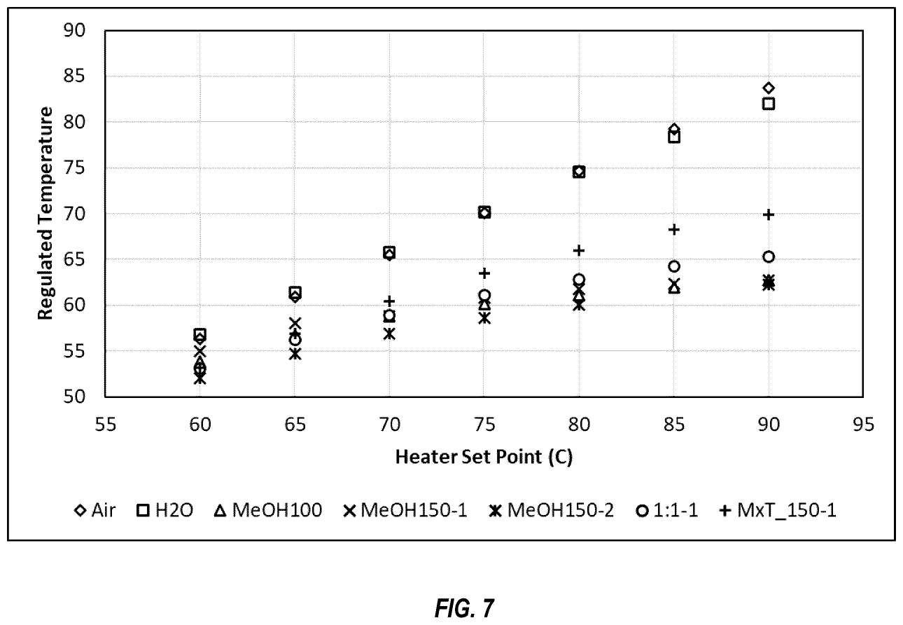

Multiple temperature regulation devices were constructed to test various phase change materials. Testing was performed by placing the temperature regulation devices between two aluminum plates. The bottom plate was heated with a resistive element. The temperature of both the top and bottom plates were monitored with thermistors. Results are shown in FIG. 7.

In FIG. 7, “Air” means liquid phase change material was omitted, “H2O” means water, “MeOH100” means methanol at 100 μL, “MeOH150” means methanol at 150 μL, “MxT_150” means a denatured alcohol (ethanol with 1 wt. % to 15 wt. % of each of methanol and isopropanol), and “1:1” means a 1:1 mixture of methanol and denatured alcohol.

As shown, “Air” and “H2O” did not provide any temperature regulation at the temperatures tested. The devices that used methanol were effective, but slightly higher regulated temperatures such as exhibited by the “MxT” and “1:1” embodiments may be preferred in certain applications.

### Example 4

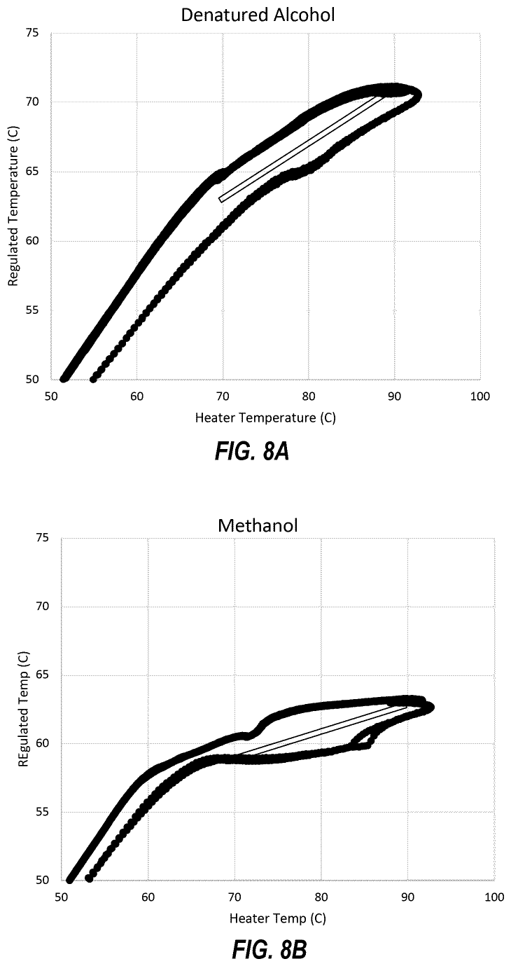

FIGS. 8A and 8B show temperature response curves for the MxT (denatured alcohol) and MeOH150 containing temperature regulation devices, respectively, using a testing setup as in Example 3. The “set point” was determined as the regulated temperature when the heater temperature was between 70 and 90 degrees Celsius, and is indicated in the Figures by the bars.

As shown, the denatured alcohol embodiment exhibited a higher, more preferred set point range. That is, a greater portion of the set point range lied within the target range of 60 to 70 degrees Celsius. However, the methanol embodiment was better able to “cut off” excessive temperatures (e.g., those exceeding about 70 degrees Celsius). Accordingly, a temperature regulation device with denatured alcohol as the phase change material may be preferred in applications where the set point has primary importance, whereas a temperature regulation device with methanol as the phase change material may be preferred in applications where cutting off excessive temperatures has primary importance. A mixture of denatured alcohol and methanol, or a mixture of ethanol and methanol, may beneficially balance both goals.

The full phase change properties of the mixture, including effects from both boiling and evaporation, can be performed to effectively regulate temperatures to assay specifications over the expected ambient temperature range the integrated devices will encounter during use.

### Example 5

Temperature regulation systems were constructed and tested. Each temperature regulation system included an oxygen-reactive pouch (available from Exothermix (College Station, TX)), a temperature regulation device containing 150 μL of “MxT” denatured alcohol as phase change material, and a microfluidics card, stacked in that order. The systems were covered in insulative foam except for cutouts for portions of the card and for oxygen ingress.

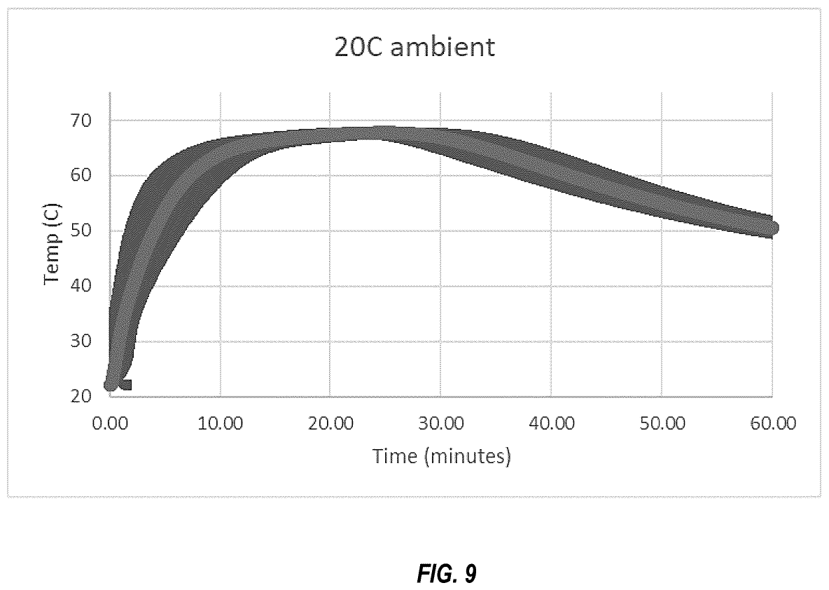

The temperature was measured at the card over time in a controlled ambient environment of 20 degrees Celsius. Results are shown in FIG. 9. As shown, the system was beneficially able to regulate the temperature within the target range of 60 to 70 degrees Celsius from about the 5 minute mark to about the 40 minute mark. Longer temperature regulation times are expected to be achievable with further optimization.

### Additional Terms & Definitions

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

The embodiments disclosed herein should be understood as comprising/including disclosed components, and may therefore include additional components not specifically described. Optionally, the embodiments disclosed herein are essentially free or completely free of components that are not specifically described. That is, non-disclosed components may optionally be completely omitted or essentially omitted from the disclosed embodiments. For example, phase change materials (including solvents), solid carriers, and/or film materials not specifically described herein may optionally be completely omitted or essentially omitted.

An embodiment that “essentially omits” or is “essentially free of” a component may include trace amounts and/or non-functional amounts of the component. For example, an “essentially omitted” component may be included in an amount no more than 2%, no more than 1%, no more than 0.1%, or no more than 0.01% by weight of relevant component (e.g., by total weight of the phase change material or solid carrier).

A composition that “completely omits” or is “completely free of” a component does not include a detectable amount of the component (i.e., does not include an amount above any inherent background signal associated with the testing instrument) when analyzed using standard coating composition analysis techniques such as, for example, chromatographic techniques (e.g., thin-layer chromatography (TLC), gas chromatography (GC), liquid chromatography (LC)), or spectroscopy techniques (e.g., Fourier transform infrared (FTIR) spectroscopy).

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

1. A temperature regulation device for regulating temperature during a biochemical reaction, comprising:
a film material arranged to form a pouch;
a solid carrier disposed within an inner cavity of the pouch; and
a phase change material disposed within the inner cavity of the pouch, the phase change material being formulated such that at least a portion undergoes phase change from a liquid to a gas while the biochemical reaction is carried out.

2. The temperature regulation device of claim 1, wherein the film comprises a polyester film.

3. The temperature regulation device of claim 2, wherein the film comprises biaxially oriented polyethylene terephthalate (BoPET).

4. The temperature regulation device of claim 1, wherein the phase change material comprises a solvent.

5. The temperature regulation device of claim 4, wherein the solvent comprises methanol, ethanol, isopropanol, or combination thereof.

6. The temperature regulation device of claim 4, wherein the solvent has a boiling point between about 20 and about 80 degrees Celsius.

7. The temperature regulation device of claim 4, wherein the solvent comprises a solvent mixture.

8. The temperature regulation device of claim 1, wherein the solid carrier comprises a fabric material.

9. The temperature regulation device of claim 1, wherein the device comprises about 0.25 mL to 2.0 mL of phase change material.

10. The temperature regulation device of claim 1, wherein the pouch is heat sealed.

11. The temperature regulation device of claim 1, wherein the solid carrier is sized to substantially match a planar surface area of the inner cavity.

12. The temperature regulation device of claim 1, wherein the device has a planar surface area of about 10 to about 100 square centimeters.

13. The temperature regulation device of claim 1, wherein the device has a phase change material volume to planar surface area ratio of about 2.5 μL/cm² to about 250 μL/cm².

14. A temperature regulation system, comprising:
the temperature regulation device of claim 1;
a heat source coupled to a first side of the temperature regulation device; and
a reaction chamber coupled to a second side of the temperature regulation device, wherein the reaction chamber is configured to carry out a biochemical reaction.

15. The temperature regulation system of claim 14, wherein the heat source comprises an exothermic reaction device.

16. The temperature regulation system of claim 15, wherein the exothermic reaction device is an oxygen-reactive device.

17. The temperature regulation system of claim 14, wherein the reaction chamber is configured to carry out an isothermal nucleic acid amplification reaction.

18. The temperature regulation system of claim 17, wherein the isothermal nucleic acid amplification reaction is LAMP or RT-LAMP.

19. A method for regulating temperature of a biochemical reaction, the method comprising:
providing a temperature regulation system that includes
(i) a temperature regulation device comprising a film material arranged to form a pouch, a solid carrier disposed within an inner cavity of the pouch, and a phase change material disposed within the inner cavity of the pouch;
(ii) a heat source coupled to a first side of the temperature regulation device; and
(iii); a reaction chamber coupled to a second side of the temperature regulation device, wherein the reaction chamber is configured to carry out a biochemical reaction;
activating the heat source to generate heat, wherein the phase change material of the temperature regulation device thereby undergoes a liquid to gas phase change; and
carrying out the biochemical reaction while the heat source is generating heat and while the phase change material is undergoing phase change.

20. The method of claim 19, wherein the biochemical reaction comprises an isothermal nucleic acid amplification reaction.
